(19) United States
(12) Patent Application Publication
(10) Pub. No.: US 2008/0086130 A1
(43) Pub. Date: Apr. 10, 2008

(54) TORSIONALLY STABLE FIXATION

(75) Inventors: Matthew Lake, Carlsbad, CA (US); Michael Mahoney, Middletown, RI (US); Timothy Beardsley, Kingston, MA (US); Christopher Sicvol, Boston, MA (US); Christopher Ramsay, West Wareham, MA (US); Erin Dupak, Fall River, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 11/539,302

(22) Filed: Oct. 6, 2006

(51) Int. Cl. A61B 17/58

(52) U.S. Cl. 606/61

(57) ABSTRACT

Methods and devices are provided for spinal fixation. In one exemplary embodiment, the methods and devices provide a spinal fixation system that can include a spinal connector which can be disposed within a recess in a head of a bone anchor. The spinal connector can have a variety of configurations. The methods and devices are particularly useful for unilateral fixation, in which one or more levels of the spine are stabilized along a single lateral side of the spine.

### FIELD OF THE INVENTION

The present invention relates generally to methods and devices for spinal fixation.

### BACKGROUND OF THE INVENTION

Spinal deformities, which include rotation, angulation, and/or curvature of the spine, can result from various disorders, including, for example, scoliosis (abnormal curvature in the coronal plane of the spine), kyphosis (backward curvature of the spine), and spondylolisthesis (forward displacement of a lumbar vertebra). Other causes of an abnormally shaped spine include trauma and spinal degeneration with advancing age. Early techniques for correcting such deformities utilized external devices that applied force to the spine in an attempt to reposition the vertebrae. These devices, however, resulted in severe restriction and in some cases immobility of the patient. Furthermore, current external braces have limited ability to correct the deformed spine and typically only prevent progression of the deformity. Thus, to avoid this need, doctors developed several internal fixation techniques to span across multiple vertebrae and force the spine into a desired orientation.

To fix the spine, surgeons attach one or more fixation elements (typically rods or plates) to the spine at several fixation sites to correct and stabilize the spinal deformity, prevent reoccurrence of the spinal deformity, and stabilize weakness in trunks that results from degenerative discs and joint disease, deficient posterior elements, spinal fracture, and other debilitating problems. Bone screws are typically used to anchor the spinal rods or plates at the various fixation sites. Once anchored, the rod-based systems are under stress and subjected to significant forces, known as cantilever pullout forces. As a result, surgeons are always concerned about the possibility of the implant loosening or the bone screws pulling out of the bone. Thus, surgeons generally seek to attach implants in the most secure and stable fashion possible while at the same time addressing a patient's specific anatomy.

Most current fixation procedures utilize two spinal rods anchored along opposed lateral sides of the spinal column. Fixation at a single level of the spine typically requires four bone screws. As a result, two incisions are often made in each lateral side of the spine at each fixation level to provide access for inserting the bone screws. In order to avoid the need to create multiple incisions and to reduce the operative and post-operative recovery time, recent fixation trends have utilized unilateral fixation, where only one spinal rod is anchored at various fixation sites along one lateral side of the spine. Because the natural forces through the spine are centered down the middle of the spine, unilateral fixation constructs must be designed to counteract the offset forces. The excess stress applied to current unilateral constructs tend to weaken the bone screws, causing them to rotate within the pedicle, and can weaken the strength of the rod itself.

Accordingly, there is a need in this art for improved methods and devices for spinal fixation, and particularly for unilateral fixation.

### SUMMARY OF THE INVENTION

The present invention generally provides methods and devices for spinal fixation. In one embodiment, a spinal fixation system is provided and includes a bone anchor having a head with a recess formed therein and configured to seat a spinal connector, and a bone-engaging member extending distally from the head and configured to engage bone. The system can also includes a spinal connector that is disposable within the recess of the head of the bone anchor. The recess and the spinal connector can include a plurality of ridges that interlock to prevent rotation of the spinal connector relative to the head of the bone anchor.

In one embodiment, the plurality of ridges can be formed adjacent to the terminal end of the spinal connector. In another embodiment, the spinal connector can be in the form of a substantially rigid spinal fixation rod and the plurality of ridges can be formed from longitudinal ridges extending along a portion of the spinal fixation rod. In a further embodiment, the recess of the head of the bone anchor can be substantially U-shaped with an open proximal portion and a closed distal portion, and the plurality of ridges can be formed in the closed distal portion.

In another embodiment, a spinal fixation system is provided and includes a bone anchor having a head with a recess formed therein. The head can have a bone engaging member extending distally therefrom that is configured to engage bone. The system can further include first and second spinal connectors that are configured to be positioned along a portion of a spinal column and disposed within the recess in the head of the bone anchor. The spinal connectors can have a variety of configurations. In one exemplary embodiment, the first spinal connector can be disposed within a closed distal portion of the recess in the head of the bone anchor, and the second spinal connector can be disposed in an open proximal portion of the recess. The disposition of the first and second spinal connectors in the recess of the head of the bone anchor can be such that they are substantially parallel to one another.

In another embodiment, a spinal fixation system is provided and can include at least one bone anchor that is adapted to be implanted in bone. The spinal fixation system can further include an elongate rigid member which can be configured to be positioned along a spinal column and can be coupled to the bone anchor. The elongate rigid member can have a variety of cross-sectional shapes, including an I-shaped cross-section, a substantially square cross-sectional shape, and a circular cross-sectional shape. The elongate rigid member can also be solid, or it can include an inner lumen extending therethrough between first and second ends thereof.

Methods for spinal fixation are also provided and in one embodiment the method can include implanting at least one bone anchor in at least one vertebra, and positioning a spinal connector within a recess formed in a head of the bone anchor. A plurality of ridges can be formed on the spinal connector and in the recess, and the ridges can interlock to prevent rotation of the spinal connector about a longitudinal axis of the spinal connector relative to the head of the bone anchor. The method can further include applying a locking mechanism to the head of the bone anchor to lock the spinal connector within the recess.

In another embodiment, a method for spinal fixation is provided and can include implanting at least one bone anchor in at least one vertebra, and positioning a first spinal connector within a recess formed in a head of the at least one bone anchor such that the first spinal connector extends along a portion of a spinal column. A second spinal connector can be positioned within the recess formed in the head of the at least one bone anchor such that the second spinal connector is seated on the first spinal connector and extends along a portion of the spinal column. The method can also include applying a locking mechanism to the head of the at least one bone anchor to lock the first and second spinal connectors within the recess formed in the head.

In another embodiment, a method for spinal fixation is provided and can include implanting first and second bone anchors in adjacent vertebrae of a spine, and coupling opposed terminal ends of a spinal connector to the first and second bone anchors. The opposed terminal ends can extend through the first and second bone anchors in a direction substantially transverse to a longitudinal axis of the spine such that a curved portion formed between the opposed terminal ends of the spinal connector extends in a medial-lateral direction to enhance torsional stiffness of the spinal connector. In one embodiment, the curved portion can include a section that extends substantially parallel to a longitudinal axis of the spine.

In another embodiment a method for spinal fixation is provided and can include implanting first and second bone anchors in adjacent vertebrae of a spine, and coupling opposed terminal ends of a spinal connector to the first and second bone anchors. In one embodiment, the spinal connector can include a curved portion formed between the opposed terminal ends of the spinal connector that extends in an anterior-posterior direction to enhance torsional stiffness of the spinal connector.

### BRIEF DESCRIPTION OF THE DRAWINGS

The invention will be more fully understood from the following detailed description taken in conjunction with the accompanying drawings, in which:

FIG. 1A is a perspective view of one embodiment of a spinal connector having a square cross-sectional shape;

FIG. 1B is a perspective view of one embodiment of a spinal connector having an I-shaped cross-section;

FIG. 1C is a perspective view of one embodiment of a spinal connector having a circular cross-sectional shape with an inner lumen extending therethrough between first and second ends;

FIG. 1D is a perspective view of one embodiment of a spinal connector having a substantially square cross-sectional shape;

FIG. 1E is a perspective view of one embodiment of a spinal connector having a square cross-sectional shape with an inner lumen extending therethrough between first and second ends;

FIG. 1F is a perspective view of one embodiment of a spinal connector having a substantially hourglass-shaped cross-section;

FIG. 2A is a perspective view of the spinal connector of FIG. 1A coupled to first and second bone anchors implanted in adjacent vertebrae;

FIG. 2B is a perspective view of the spinal connector of FIG. 1D coupled to first and second bone anchors implanted in adjacent vertebrae;

FIG. 3 is a perspective view of another embodiment of a spinal fixation system having a spinal connector with a plurality of ridges formed thereon for engaging corresponding ridges formed in a bone anchor;

FIG. 4 is a perspective view of yet another embodiment of a spinal connector coupled to first and second bone anchors implanted in adjacent vertebrae, showing a plurality of pores formed in the spinal connector for promoting bone in-growth;

FIG. 5A is a perspective view of an another embodiment of a spinal fixation system having first and second spinal connectors coupled to first and second bone anchors implanted in adjacent vertebrae, showing the first and second spinal connectors in a stacked configuration;

FIG. 5B is a perspective view of yet another embodiment of a spinal fixation system having first and second spinal connectors coupled to first and second bone anchors implanted in adjacent vertebrae, showing the first and second spinal connectors in a parallel configuration;

FIG. 6A is a perspective view of another embodiment of a spinal connector coupled to first and second bone anchors implanted in adjacent vertebrae, showing the spinal connector having a curved portion that extends in a medial-lateral direction;

FIG. 6B is perspective view of yet another embodiment of a spinal connector coupled to first and second bone anchors implanted in adjacent vertebrae, showing the spinal connector having a curved portion that extends in an anterior-posterior direction;

FIG. 6C is another perspective view of one embodiment of a spinal connector coupled to first and second bone anchors implanted in adjacent vertebrae, showing the spinal connector having a curved portion that extends in a medial-lateral direction; and

FIG. 6D is a perspective view of yet another embodiment of a spinal connector coupled to first and second bone anchors implanted in adjacent vertebrae, showing the spinal connector having a longitudinal member that extends substantially parallel to a longitudinal axis of the spine.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for spinal fixation, and in particular, for increasing the torsional stability of a spinal connector, such as a spinal rod, relative to one or more bone anchors, such as bone screws. An increased torsional stability is particularly desirable with unilateral fixation, in which one or more levels of the spine are stabilized along only one lateral side of the spine. Since the natural forces through the spine are centered down the middle of the spine, the enhanced torsional stability of the devices provided herein will help to counteract the offset and twisting forces applied to the spinal fixation system. A person skilled in the art will appreciate that, while the methods and devices are described in connection with unilateral fixation, the methods and devices can be used in various procedures in which it is desirable to provide enhanced torsional stability between a spinal fixation device and a bone anchor or other spinal instrumentation.

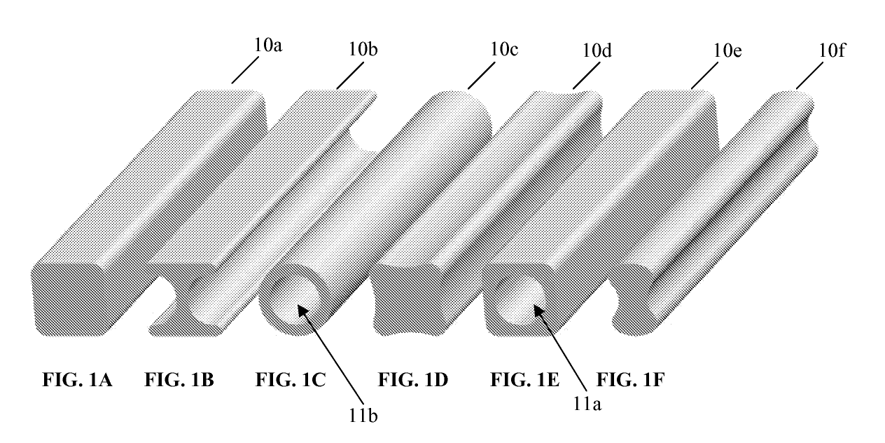

FIGS. 1A-1F illustrate one technique for enhancing the torsional stability of a spinal connector. In this embodiment, the cross-sectional geometry of a spinal rod is modified to increase the bending resistance and thus the torsional stability. The cross-sectional shape can also enhance the strength of the interface between the spinal rod and a bone anchor, preventing rotation and other movement between the components. While various shapes can be used, FIG. 1A illustrates a spinal rod 10a having a square cross-sectional shape. In another embodiment, as shown in FIG. 1D, the spinal rod 10d can have a square cross-sectional shape with concave outer walls. The square configuration will render the rod more difficult to bend as compared to a standard cylindrical rod, and it will also prevent rotation between the rod and a bone anchor when the two components are mated. In another embodiment, shown in FIG. 1C, the spinal rod 10c can have a circular cross-sectional shape with an inner lumen 11b extending therethrough between first and second ends thereof. Similarly, as shown in FIG. 1E, the spinal rod 10e can have a square cross-sectional shape with an inner lumen 11a extending therethrough between first and second ends thereof. The inner lumens 11a, 11b can have, for example, a circular shape or any other shape. Spinal rods having an inner lumen provide enhanced bending stiffness, thereby ensuring greater torsional stability of a spinal connector. In yet another embodiment, the spinal rod 10b can have an I-shaped cross-section as shown in FIG. 1B, or a substantially hourglass-shaped cross-section as shown in FIG. 1F. Spinal rods with I-shaped or hourglass-shaped cross-sections demonstrate a superior resistance to bending over conventional circular rods, as well as ensuring a more secure placement of the spinal rod within a bone anchor. That is, when oriented properly, the rod will have an increased bending strength in one direction. A person skilled in the art will appreciate that a variety of other cross-sectional shapes can be used to increase the bending strength of the rod.

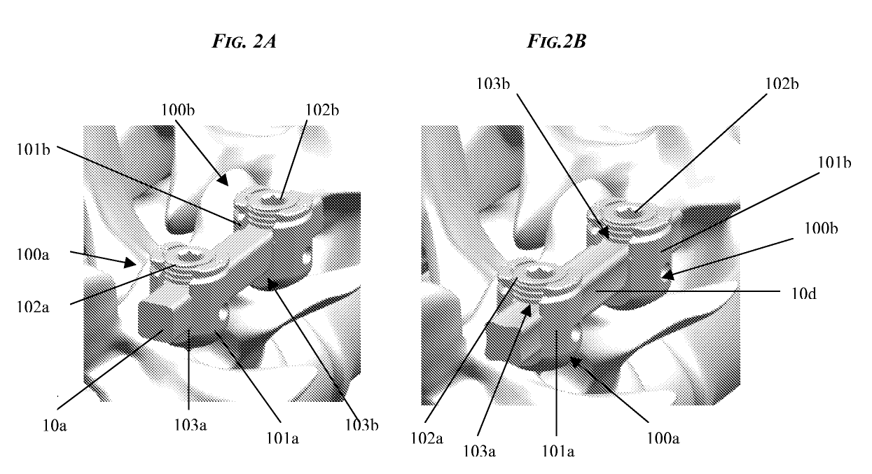

FIGS. 2A-2B illustrate the spinal rods of FIGS. 1A and 1D in use with a spinal fixation system. As shown, the spinal rods 10a, 10d can be anchored to one or more vertebra by mating the spinal rods 10a, 10d to one or more bone anchors, such as bone screws 100a, 100b, implanted in adjacent vertebrae such that the spinal rods 10a, 10d extend longitudinally along a lateral side of a patient's spinal column. In this embodiment, while bone screws are shown various other bone anchors, such a spinal hooks, plates, etc., can be used. Referring to FIG. 2A, the spinal rod 10a is coupled to a first bone screw 100a and a second bone screw 100b. The first bone screw 100a generally includes a rod-receiving head 101a having a U-shaped recess 103a formed therein for seating the spinal rod 10a, and a threaded shank (not shown) extending distally from the rod-receiving head 101a. Similarly, the second bone screw 100b can include a rod-receiving head 101b having a U-shaped recess 103b formed therein for seating the spinal rod 10a, and a threaded shank (not shown) extending distally from the rod-receiving head 101b. The spinal fixation system of this embodiment can further include first and second locking mechanisms 102a, 102b that mate to the heads 101a, 101b of the bone screws 100a, 100b to lock the spinal rod 10a within the recesses 103a, 103b. FIG. 2B similarly depicts spinal rod 10d anchored to first and second adjacent vertebrae using the bone screws 100a, 100b and locking mechanisms 102a, 102b as described with reference to FIG. 2A.

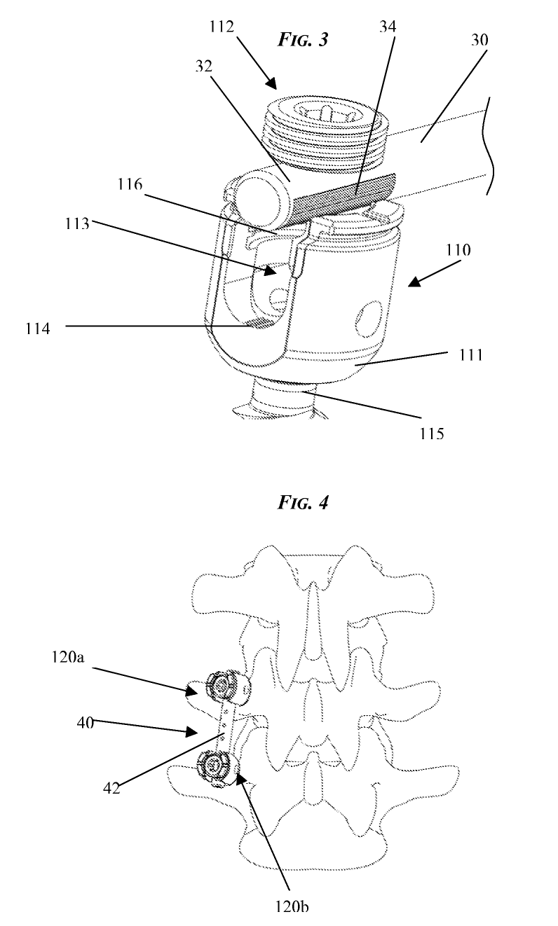

FIG. 3 illustrates another technique for providing unilateral spinal fixation. In this embodiment, the spinal fixation system includes a spinal rod 30 and a bone anchor, e.g. a bone screw 110, that are modified to include surface features which enhance the coupling between the spinal rod 30 and the bone screw 110. The improved interface lends greater strength and torsional stability to the spinal fixation system, thereby increasing the resistance to the natural offset forces of the spine. As shown in FIG. 3, the spinal rod 30 can have a series of ridges 34 formed adjacent to a terminal end 32 thereof. The bone anchor, shown herein in the form of a bone screw 110, can include a head 111 having opposed arms that define a U-shaped rod-receiving recess 113 therebetween, and a threaded shank 115 that extends distally from the head 111. The U-shaped recess 113 of the head 111 can include an open proximal portion and a closed distal portion. A series of ridges 114 can be formed within the closed distal portion of the recess 113.

In use, the spinal rod 30 is coupled to the bone screw 110 by seating the spinal rod 30 within the recess 113 of the bone screw 110. Once the spinal rod 30 is seated within the bone screw 110, the ridges 34 on the spinal rod 30 mechanically interlock with the ridges 114 on the bone screw 100. The interlocking of the two sets of ridges prevents rotation of the spinal rod 30 about a longitudinal axis of the rod 30 relative to the head 111 of the bone screw 110, thus enhancing the stability of the spinal fixation system. FIG. 3 also illustrates a threaded locking mechanism 112 that mates with corresponding threads 116 formed in the head 111 of the bone screw 110 to lock the spinal rod 30 within the recess 113 of the head 111, and thereby further strengthen the interlocking of the spinal rod 30 with the bone screw 110. A person skilled in the art will appreciate that other surface features, such as teeth, pins, etc. can be used instead of grooves, and that one or both components can include surface features.

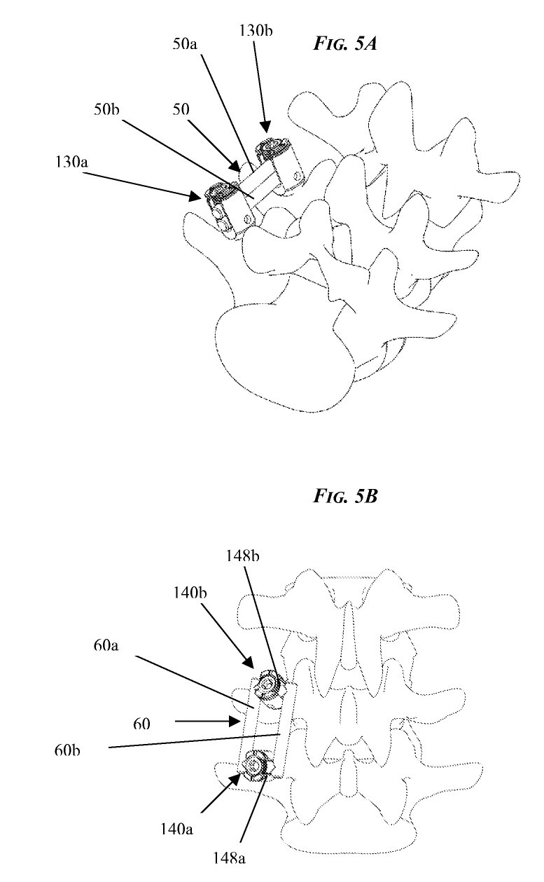

FIG. 4 illustrates yet another technique for providing unilateral spinal fixation. In this embodiment, the spinal fixation system includes a spinal connector, such as an elongate rigid rod, which is modified to include a fenestrated portion to promote bone in-growth. In particular as depicted in FIG. 4, the spinal rod 40 can include perforations 42 covering either all or a part of the surface of the spinal rod 40. The rod 40 can be hollow such that the perforations 42 extend into an inner lumen, or the rod 40 can be solid and the perforations 42 can be in the form of blind bores or tunnels extending into or through the rod 40. One skilled in the art will appreciate that the spacing, sizing, and formation of the perforations 42 can vary.

In use, the spinal rod 40 can be coupled to first and second bone screws and implanted into one or more vertebrae. Once the spinal rod 40 is anchored to one or more vertebrae, a substance, such as cement or a bone growth material, can be injected into the spinal rod 40. The substance seeps into the surrounding bony anatomy through the perforations 42 and promotes fusion of the spinal rod 40 to the vertebrae, thereby ensuring torsional stability of the spinal fixation system. The use of a perforated spinal rod in a spinal fixation system provides another advantage by permitting the introduction of osteobiologics into the surrounding bony anatomy.

In yet another unilateral spinal fixation technique, a spinal connector, such as an elongate rigid rod, can be modified by coating its surface with a substance to promote fusion of the spinal rod with the surrounding bony anatomy. The substance can be, for example, a bone growth stimulating substance such as hydroxyapatite. One skilled in the art will appreciate that a variety of biocompatible substances can be used to coat the spinal connector of the present invention. In use, the coated spinal connector can be implanted into one or more vertebrae to promote the surrounding bone to grow onto and fuse with its surface, thereby providing a stronger construct and improved fixation.

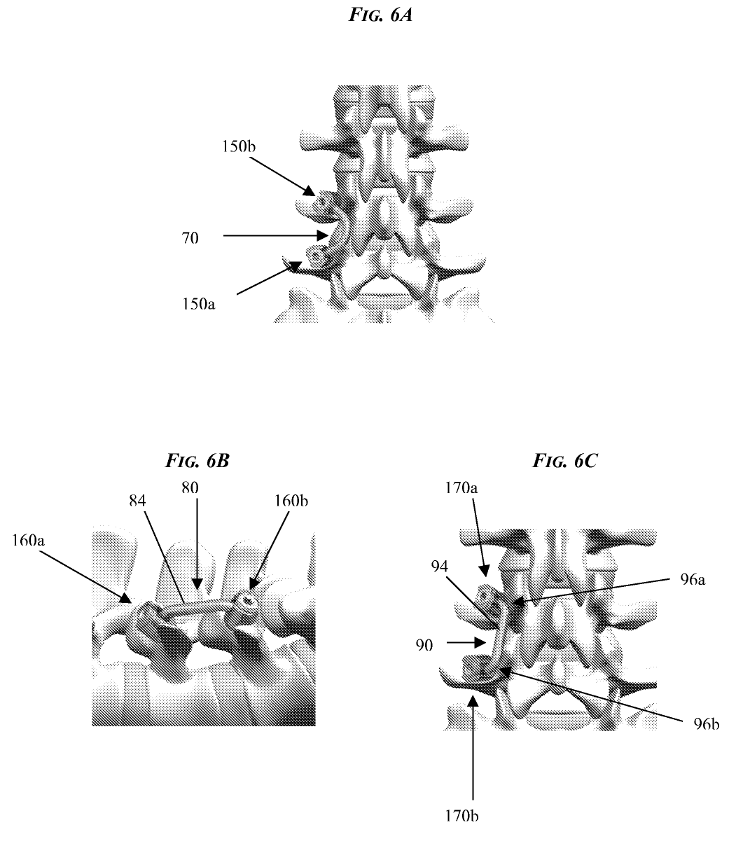

FIGS. 5A-5B illustrate another technique for providing unilateral spinal fixation. In this embodiment, a spinal fixation system is modified to anchor two or more spinal rods adjacent to one another along a lateral side of the spine, thereby providing the spinal fixation system with an improved torsional stability. As shown, a pair of spinal rods 50, 60 can be anchored to one or more vertebrae, preferably by mating the spinal rods 50, 60 to one or more bone screws implanted in one or more adjacent vertebrae, such that the spinal rods 50, 60 extend longitudinally along a lateral side of a patient's spinal column. Referring to FIG. 5A, the pair of spinal rods 50 are shown coupled to a first bone screw 130a and a second bone screw 130b. The first and second bone screws 130a, 130b are similar to the bone screws previously described with respect to FIG. 3. As shown, the first spinal rod 50a can be seated in the closed distal portion of the recess in the first and second bone screws 130a, 130b, and the second spinal rod 50b can be seated in the open proximal portion of the recesses, such that rod 50b is in a stacked position relative to rod 50a. In another embodiment, as illustrated in FIG. 5B, a pair of spinal rods 60 can be coupled to first and second bone screws 140a, 140b such that a first spinal rod 60a is spaced apart from, but parallel to, a second spinal rod 60b. The first and second rods 60a, 60b can optionally be mated to one another using one or more connecting elements or struts extending therebetween. FIG. 5B illustrates a first connecting element 148a extending between a first terminal end of each rod 60a, 60b, and a second connecting element 148b extending between a second terminal end of each rod 60a, 60b. The connecting elements 148a, 148b can be used to mate the rods 60a, 60b to the bone screws 140a, 140b in a parallel orientation. In particular, FIG. 5B illustrates the first connecting element 148a seated in the head of the first bone screw 140a and the second connecting element 148b seated in the head of the second bone screw 140b. The rods 60a, 60b are positioned on opposed sides of each bone screw 140a, 140b. As a result, the pair of rods 60 provide increased torsional stability. One skilled in the art will appreciate that the spinal rods 60 can be coupled to the bone screws 140a, 140b in a variety of ways to achieve a parallel configuration.

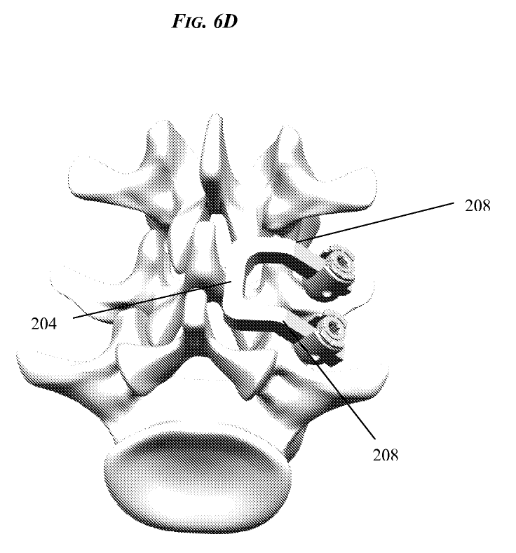

FIGS. 6A-6D illustrate another technique for enhancing the torsional stability of a spinal connector. In this embodiment, a spinal rod is modified to have a curved portion thereby increasing the bending resistance and thus the torsional stability. In one embodiment, as shown in FIG. 6A, a spinal rod 70 having a curved portion is coupled to first and second bone screws 150a, 150b that are implanted in adjacent vertebrae, such that the curved portion of the spinal rod 70 extends in a direction substantially transverse to a longitudinal axis of the spine. In particular, the opposed terminal ends of the rod 70 are anchored on a lateral side of the spine, and the curved portion of the rod 70 is curved toward a central axis of the spine. In another embodiment, as shown in FIG. 6B, rather than having the curved portion extend toward the central axis of the spine, a spinal rod 80 is provided having a curved portion extending in a substantially anterior-posterior plane relative to a longitudinal axis of the spine. In particular, the opposed terminal ends of the rod 80 are mated to first and second bone screws 160a, 160b implanted in adjacent vertebrae, and a mid-portion 84 of the rod 80 extends in a posterior direction relative to the spinal column. FIG. 6C depicts another spinal rod 90 having a curved portion coupled to first and second bone screws 170a, 170b that are implanted in adjacent vertebrae such that the curved portion of the spinal rod 90 extends in a direction substantially transverse to a longitudinal axis of the spine. In this embodiment, rather than having an arc-shaped curve as shown in FIG. 6A, the spinal rod 90 includes two bend zones 96a, 96b formed a distance apart from the terminal ends of the rod 90. As a result, a mid-portion 94 of the rod 90 extending between the bends zones 96a, 96b is substantially linear and is longitudinally aligned with the longitudinal axis of the spine. This allows the mid-portion 94 to be positioned closer to the center of the spine, thus allowing the forces to be distributed near the center of the spinal column. This is particularly advantages for unilateral fixation. In yet another embodiment, shown in FIG. 6D, the curved portion of the spinal rod 200 can be substantially U-shaped, such that it has opposed arms 208 and a substantially linear central portion 204. In use, the spinal rod 200 can be anchored to adjacent vertebrae as shown, such that the opposed arms 208 of the spinal rod 200 extend substantially perpendicular to the central axis of the spinal column, and the linear central portion 204 extends parallel to the central axis of the spinal column. The central portion 204 can also be positioned adjacent to the center of the spinal column to allow the forces to be distributed near the center of the spine.

The present invention also provides exemplary methods for unilateral fixation. In one exemplary embodiment, a spinal fixation system can be implanted in a patient's spine. In particular, a bone anchor, such as a bone screw, can be implanted in each vertebra to be affixed on one lateral side of the spine. The opposed lateral side of each vertebra can remain un-affixed. A spinal rod can be mated to the bone anchors implanted in the lateral side of the spine such that the spinal rod extends longitudinally along the lateral side of the spine. In order to provide torsional stability and to counteract any offset forces received as a result of affixing only one lateral side of the spine, the spinal rod and/or bone anchor can include various features such as those previously described herein. Once the spinal rod is coupled to bone screws, and the spinal fixation system is implanted in adjacent vertebrae, a locking mechanism can be applied to a head of the bone screw to lock the spinal rod within the head. The methods described herein result in an enhanced torsional stability of the spinal rod and/or bone anchors, resulting in a reduction of the twisting forces experienced by the spinal fixation system when implanted in a spine, and thus counteracting the natural offset forces of the spine.

The methods and devices disclosed herein can also be used in conjunction with other techniques for enhancing unilateral fixation. By way of non-limiting example, commonly-owned U.S. Provisional Patent Application No. 60/828,428 filed on even date herewith and entitled “Improved Bone Screw Fixation,” by Dupak et al. (Atty. Docket No. 101896-494PROV (DEP5766)), which is hereby incorporated by reference in its entirety, discloses various exemplary spinal anchors having an anti-rotation mechanism located on a bone anchor for preventing rotation of at least a portion of the bone anchor relative to bone. U.S. patent application Ser. No. 11/539,295 filed on even date herewith and entitled “Unilateral Placement,” by Mahoney et al. (Atty. Docket No. 101896-467 (DEP5760)), which is hereby incorporated by reference in its entirety, also discloses various exemplary spinal anchors having one or more points of fixation located adjacent to a first fixation point at which a bone anchor is implanted in bone. The various spinal connectors disclosed herein can be used in combination with the above-referenced spinal anchors to provide a more secure spinal fixation construct, which is particularly useful during unilateral fixation. A person skilled in the art will appreciate, however, that the spinal connectors disclosed herein can be used with virtually any bone anchor known in the art.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A spinal fixation system, comprising:
a bone anchor having a head with a recess formed therein and configured to seat a spinal connector, and a shank extending distally from the head and configured to engage bone; and
a spinal connector disposable within the recess in the head;
wherein the recess and the spinal connector include a plurality of ridges that interlock to prevent rotation of the spinal connector relative to the head of the bone anchor.

2. The system of claim 1, wherein the plurality of ridges are formed adjacent to a terminal end of the spinal connector.

3. The system of claim 1, wherein the spinal connector comprises a substantially rigid spinal fixation rod, and wherein the plurality of ridges are formed from longitudinal ridges extending along a portion of the spinal fixation rod.

4. The system of claim 1, wherein the plurality of ridges are formed within the recess of the head.

5. The system of claim 1, wherein the recess is substantially U-shaped with an open proximal portion and a closed distal portion, and wherein the plurality of ridges are formed in the closed distal portion of the recess.

6. A method for spinal fixation, comprising:
implanting at least one bone anchor in at least one vertebra; and
positioning a spinal connector within a recess formed in a head of the bone anchor such that a plurality of ridges formed on the spinal connector and in the recess interlock to prevent rotation of the spinal connector about a longitudinal axis of the spinal connector relative to the head of the bone anchor.

7. The method of claim 6, further comprising applying a locking mechanism to the head of the bone anchor to lock the spinal connector within the recess.

8. A spinal fixation system, comprising:
a bone anchor having a head with a recess formed therein, and a shank extending distally from the head and configured to engage bone; and
first and second spinal connectors configured to be positioned along a portion of a spinal column and disposed within the recess in the head of the bone anchor.

9. The system of claim 8, wherein the first spinal connector is disposed within a distal portion of the recess in the head of the bone anchor, and the second spinal connector is disposed within a proximal portion of the recess in the head of the bone anchor such that the first and second spinal connectors are substantially parallel to one another.

10. A spinal fixation system, comprising:
at least one anchor adapted to be implanted in bone; and
an elongate rigid member configured to be positioned along a spinal column and to couple to the at least one anchor, the elongate rigid member having an I-shaped cross-section.

11. A spinal fixation system, comprising:
at least one anchor adapted to be implanted in bone; and
an elongate rigid member configured to be positioned along a spinal column and to couple to the at least one anchor, the elongate rigid member having a substantially square cross-sectional shape with an inner lumen extending therethrough between first and second ends thereof.

12. A spinal fixation system, comprising:
at least one anchor adapted to be implanted in bone; and
an elongate rigid member configured to be positioned along a spinal column and to couple to the at least one anchor, the elongate rigid member having a circular cross-sectional shape with an inner lumen extending therethrough between first and second ends thereof.

13. A method for spinal fixation, comprising:
implanting at least one bone anchor in at least one vertebra;
positioning a first spinal connector within a recess formed in a head of the at least one bone anchor, the first spinal connector extending along a portion of a spinal column; and
positioning a second spinal connector within the recess formed in the head of the at least one bone anchor such that the second spinal connector is seated on the first spinal connector and extends along a portion of the spinal column.

14. The method of claim 13, further comprising applying a locking mechanism to the head of the at least one bone anchor to lock the first and second spinal connectors within the recess formed in the head.

15. A method for spinal fixation, comprising:
implanting first and second bone anchors in adjacent vertebrae of a spine;
coupling opposed terminal ends of a spinal connector to the first and second bone anchors, the opposed terminal ends extending through the first and second bone anchors in a direction substantially transverse to a longitudinal axis of the spine such that a curved portion formed between the opposed terminal ends of the spinal connector extends in a medial-lateral direction to enhance torsional stiffness of the spinal connector.

16. The method of claim 15, wherein the curved portion includes a section that extends substantially parallel to a longitudinal axis of the spine.

17. A method for spinal fixation, comprising:
implanting first and second bone anchors in adjacent vertebrae of a spine;
coupling opposed terminal ends of a spinal connector to the first and second bone anchors, the spinal connector including a curved portion formed between the opposed terminal ends of the spinal connector that extends in an anterior-posterior direction to enhance torsional stiffness of the spinal connector.
